(19) United States
(12) Patent Application Publication
(10) Pub. No.: US 2024/0261478 A1
(43) Pub. Date: Aug. 8, 2024

(54) AUTOMATIC BREAST PUMP DEVICE

(71) Applicants: Jennifer Benson, Yonkers, NY (US); Charles Benson, Yonkers, NY (US)

(72) Inventors: Jennifer Benson, Yonkers, NY (US); Charles Benson, Yonkers, NY (US)

(21) Appl. No.: 18/106,850

(22) Filed: Feb. 7, 2023

(51) Int. Cl. A61M 1/06

(52) U.S. Cl. CPC A61M 1/067; A61M 1/064

(57) ABSTRACT

An automatic breast pump device for automatically pumping milk from a user's breast while the user sleeps and maintaining a cool environment for the extracted milk includes a housing that contains a container, a cooling unit, and a vacuum pump. A breast engagement member is fluidly coupled to the container and is attached to a garment which the user wears to position the breast engagement member on the user's breast. The device is operable to create suction in the container and draw milk from the user's breast into the container. The cooling unit keeps the container cool.

### CROSS-REFERENCE TO RELATED APPLICATIONS

### Not Applicable

### STATEMENT REGARDING FEDERALLY SPONSORED RESEARCH OR DEVELOPMENT

### Not Applicable

### THE NAMES OF THE PARTIES TO A JOINT RESEARCH AGREEMENT

### Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

### Not Applicable

### STATEMENT REGARDING PRIOR DISCLOSURES BY THE INVENTOR OR JOINT INVENTOR

### Not Applicable

### BACKGROUND OF THE INVENTION

### (1) Field of the Invention

The disclosure relates to breast pumps and more particularly pertains to a new breast pump for automatically pumping milk from a user's breast while the user sleeps and maintaining a cool environment for the extracted milk.

### (2) Description of Related Art Including Information Disclosed Under 37 CFR 1.97 and 1.98

The prior art relates to breast pumps including those which cycle automatically through a pumping sequence without intervention by a user. The prior art also discloses several garments which position a breast engagement member over the nipple of a breast of a user so that the breast pump can pump milk from the breast without the user maintaining the position of the breast engagement member. However, the prior art is silent as to a device which provides a garment for positioning a breast engagement member, pumps milk in a programmed pumping sequence without intervention by the user and cools the collected milk so that the user can sleep during pumping.

### BRIEF SUMMARY OF THE INVENTION

An embodiment of the disclosure meets the needs presented above by generally comprising a housing has an outer wall and an interior space, the interior space bounded by the outer wall. A cooling unit is coupled to the housing and is positioned within the interior space for selectively cooling the interior space. A container is positioned on a bottom panel of the outer wall and within the interior space. A vacuum pump is coupled to the housing and is positioned within the interior space. The vacuum pump includes a motor for driving the vacuum pump, and the vacuum pump is fluidly coupled to a top end of each container to selectively cause a negative pressure within each container.

A tube is coupled to and extends away from the container. The tube is flexible and has a distal end relative to the container. A breast engagement member is coupled to the distal end of the tube. The breast engagement member has a concave surface and a channel. The channel extending through the concave surface of the breast engagement member and is fluidly coupled to the tube. The concave surface is configured for sealingly engaging one of a pair of breasts of a user, and the channel is configured for receiving a nipple of the associated breast.

A garment is coupled to each breast engagement member. The garment comprises a flexible tubular panel which forms a pair of recesses on an inner side of a front portion of the garment. Each recess is laterally positioned with respect to another one of the pair of recesses, and the breast engagement member is positioned within one of the pair of recesses. The concave surface of the recess faces inwardly with respect to the garment. The garment is configured for surrounding a back and a chest of the user, and the garment is configured for positioning the concave surface of each breast engagement member in sealing engagement with one of the pair of breasts.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top front side perspective view of an automatic breast pump device according to an embodiment of the disclosure.

FIG. 2 is a front in-use view of an embodiment of the disclosure.

FIG. 3 is a side view of an embodiment of the disclosure.

FIG. 4 is a cross-section in-use view of an embodiment of the disclosure taken from the Arrows 4-4 in FIG. 2.

FIG. 5 is an in-use view of an embodiment of the disclosure.

FIG. 6 is a block diagram of an embodiment of the disclosure.

### DETAILED DESCRIPTION OF THE INVENTION

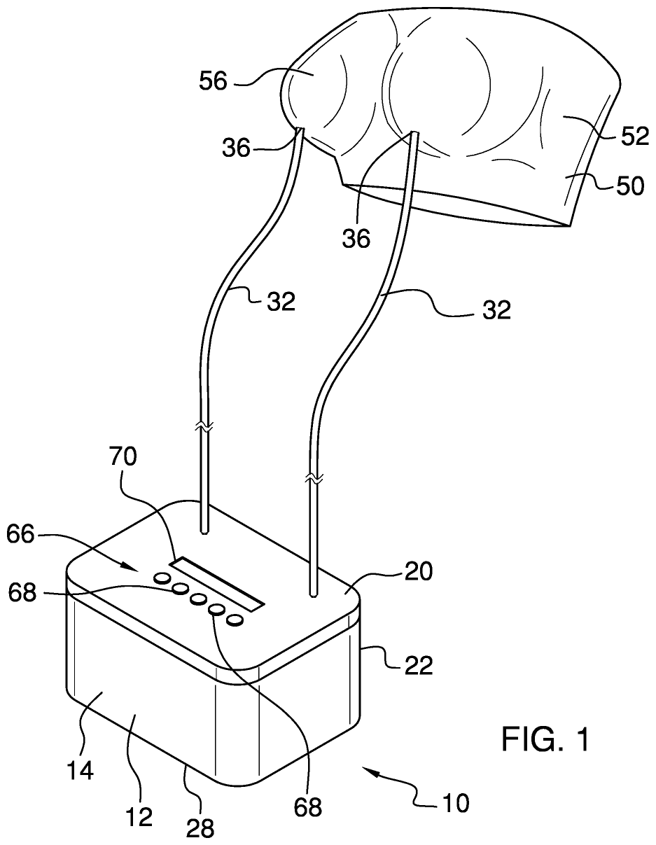

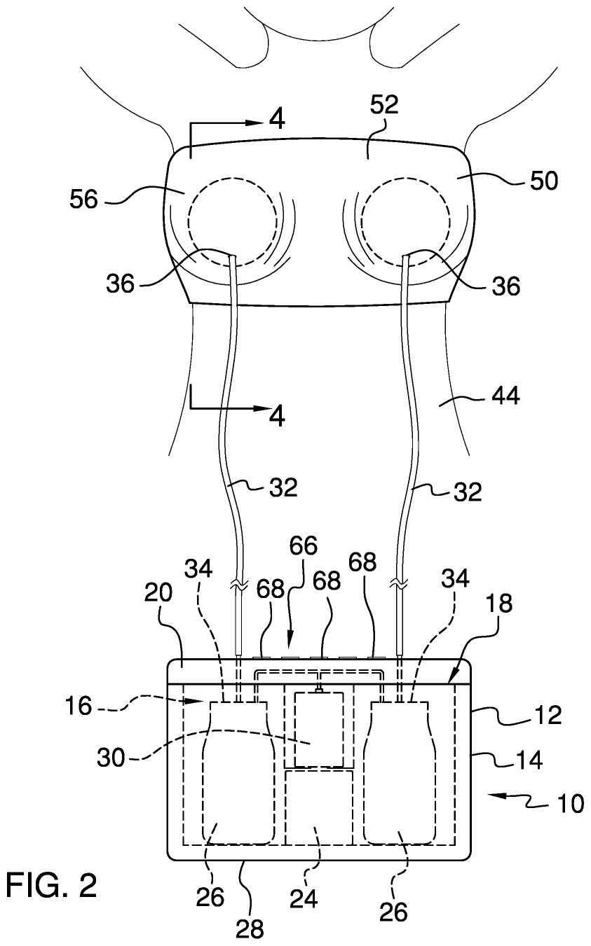

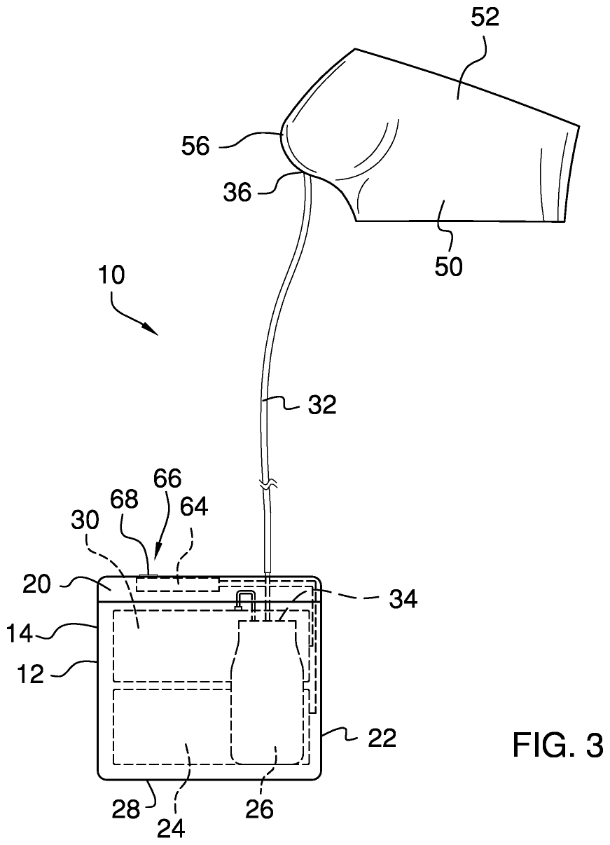

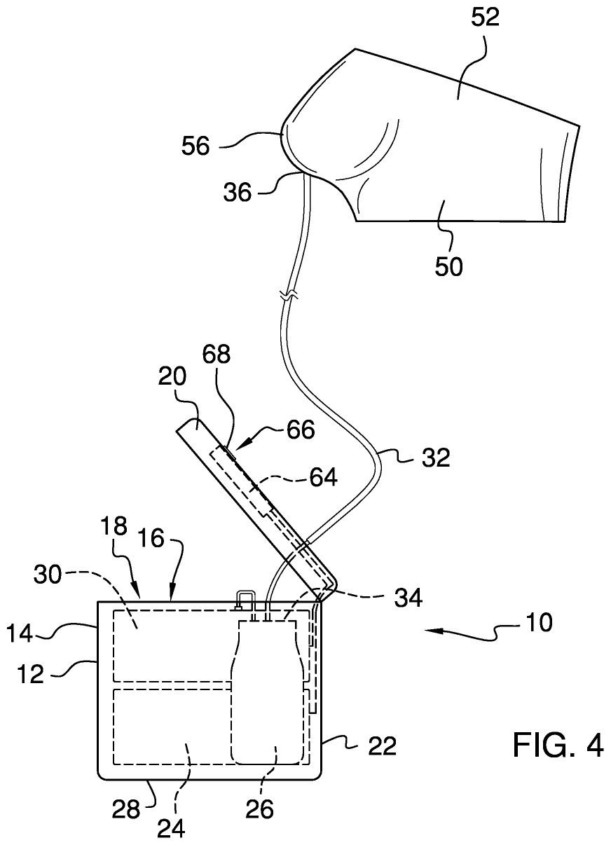

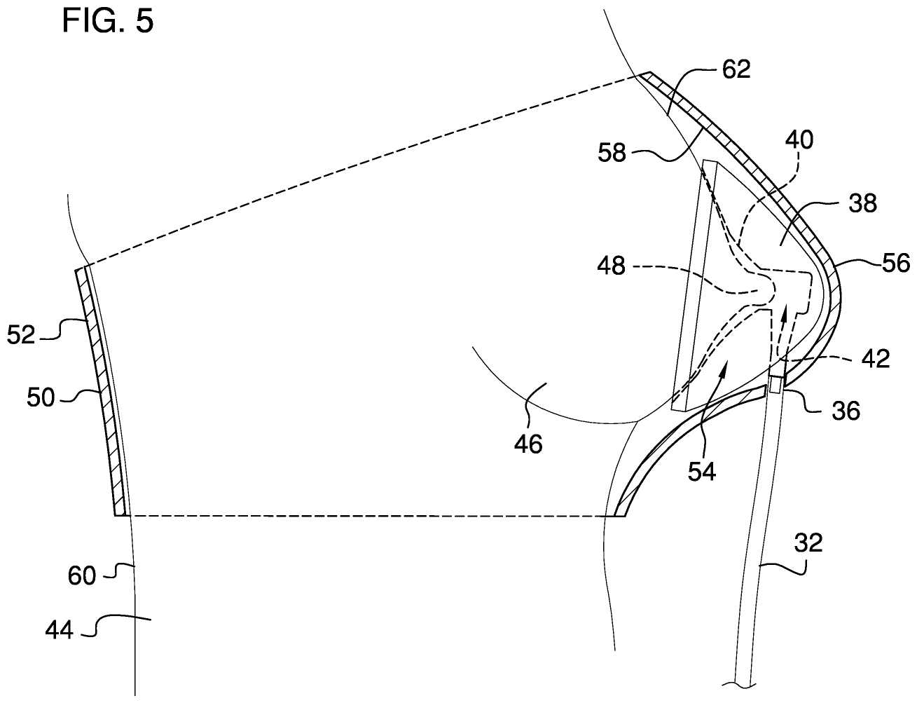

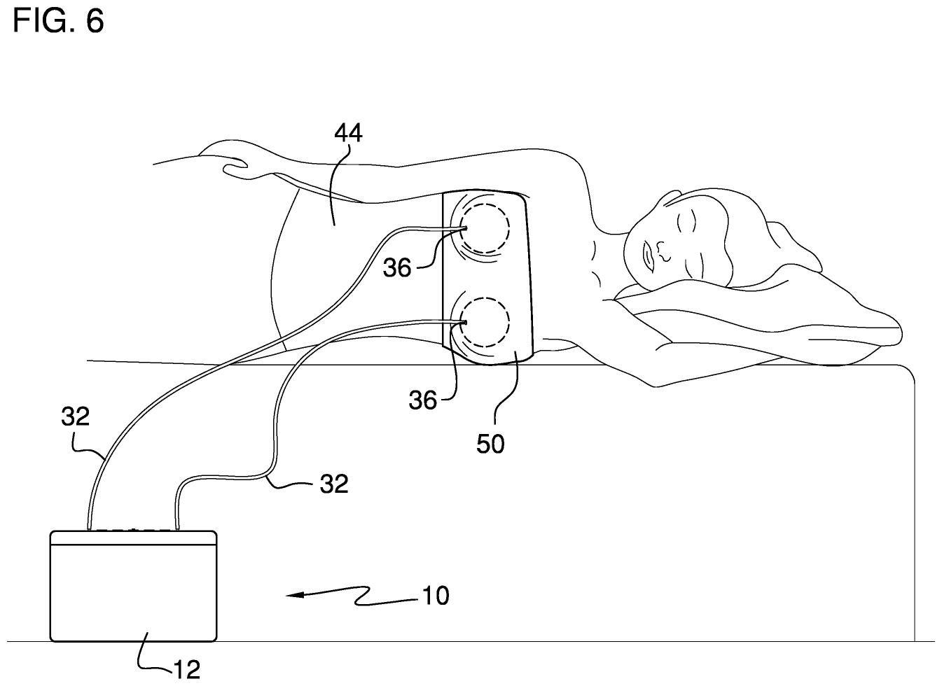

With reference now to the drawings, and in particular to FIGS. 1 through 6 thereof, a new breast pump embodying the principles and concepts of an embodiment of the disclosure and generally designated by the reference numeral 10 will be described.

As best illustrated in FIGS. 1 through 6, the automatic breast pump device 10 generally comprises a housing 12 with an outer wall 14 and an interior space 16, the interior space 16 bounded by the outer wall 14. The housing 12 has an open top 18, and the outer wall 14 comprising a thermally insulating material. A lid 20 is positionable to cover the open top 18 of the housing 12 and also comprises the thermally insulating material. The lid 20 is pivotally coupled to a back panel 22 of the outer wall 14.

A cooling unit 24 for selectively cooling the interior space 16 is coupled to the housing 12 and is positioned within the interior space 16. The cooling unit 24 comprises a refrigeration system. A pair of containers 26 is positioned on a bottom panel 28 of the outer wall 14 and within the interior space 16. Each container 26 is removable from the interior space 16. A vacuum pump 30 is coupled to the housing 12 and is positioned within the interior space 16. The vacuum pump 30 includes a motor for driving the vacuum pump 30. The vacuum pump 30 is fluidly coupled to a top end 34 of each container 26 to selectively cause a negative pressure within each container 26. The cooling unit 24 is laterally centered within the interior space 16, and each container 26 is positioned opposite another of the pair of containers 26 across the cooling unit 24. The vacuum pump 30 is positioned above the cooling unit 24 and between each one of the pair of containers 26.

Each tube 32 of a pair of tubes 32 is coupled to and extends away from an associated one of the pair of containers 26. Each tube 32 is coupled to the top end 34 of the associated container 26 and has a distal end 36 relative to the associated container 26. Each tube 32 is flexible and extends through the lid 20. Each one of a pair of breast engagement members 38 is coupled to the distal end 36 of an associated one of the pair of tubes 32. Each breast engagement member 38 has a concave surface 40 and a channel 42. The channel 42 extends through the concave surface 40 of an associated one of the pair of breast engagement members 38 and is fluidly coupled to the associated tube 32. The concave surface 40 of each breast engagement member 38 is configured for sealingly engaging an associated one of a pair of breasts 46 of a user 44. The channel 42 of each breast engagement member 38 is configured for receiving a nipple 48 of the associated breast 46.

A garment 50 comprising a flexible tubular panel 52 is coupled to each breast engagement member 38. The flexible tubular panel 52 forms a pair of recesses 54 on an inner side 58 of a front portion 56 of the garment 50, and each recess 54 is laterally positioned with respect to another one of the pair of recesses 54. Each breast engagement member 38 is positioned within an associated one of the pair of recesses 54 such that the concave surface 40 of each breast engagement member 38 facing inwardly with respect to the garment 50. Each tube 32 extends through the front portion 56 of the garment 50. The garment 50 is configured for surrounding a back 60 and a chest 62 of the user 44. The garment 50 is also configured for positioning the concave surface 40 of each breast engagement member 38 in sealing engagement with one of the pair of breasts 46.

A control circuit 64 is coupled to the lid 20 and is positioned within the interior space 16. The control circuit 64 is electrically coupled to each of the cooling unit 24 and the vacuum pump 30. The control circuit 64 includes a thermometer and is programmable to activate the cooling unit 24 to cool each container 26 to a selected temperature. The control circuit 64 is also programmable to activate the vacuum pump 30 to cause the negative pressure in each container 26 over a selected time period, at a selected frequency, and to a selected magnitude with respect to a pressure of the channel 42 of each breast engagement member 38. Each one of the selected time period, the selected frequency, and the selected magnitude may also be selectable by a user 44. An input 66 comprising a plurality of buttons 68 is coupled to the lid 20 for activating and programming the control circuit 64. A display 70 is coupled to the lid 20 for displaying an indicia associated with operation of the automatic breast pump device 10. The indicia may include options for the selected time period, the selected frequency, and the selected magnitude. The indicia may also include information such as a temperature of the interior space 16 and a length of time for which the automatic breast pump device 10 has been operation. A power source may be included and electrically coupled to the control circuit 64, and the control circuit 64 may be electrically couplable to an external power supply.

In use, the user 44 wears the garment 50 with the concave surface 40 of each breast engagement member 38 in sealing engagement with the associated breast 46 such that the channel 42 of each breast engagement member 38 receives the nipple 48 of the associated breast 46. The vacuum pump 30 and the cooling unit 24 are activated according to the selected time period, the selected frequency, and the selected magnitude. With the automatic breast pump device 10 in operation, the user 44 is free to sleep. When the user 44 awakens, the containers 26 can be removed from the interior space 16 if desired.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An automatic breast pump device comprising:
a housing having an outer wall and an interior space, said interior space bounded by said outer wall;
a cooling unit being coupled to said housing, said cooling unit being positioned within said interior space for selectively cooling said interior space;
a container being positioned on a bottom panel of said outer wall and within said interior space;
a vacuum pump being coupled to said housing, said vacuum pump being positioned within said interior space, said vacuum pump including a motor for driving said vacuum pump, said vacuum pump being fluidly coupled to a top end of each said container to selectively cause a negative pressure within each said container;
a tube being coupled to and extending away from said container, said tube having a distal end relative to said container, said tube being flexible;
a breast engagement member being coupled to said distal end of said tube, said breast engagement member having a concave surface and a channel, said channel extending through said concave surface of said breast engagement member and being fluidly coupled to said tube, said concave surface being configured for sealingly engaging one of a pair of breasts of a user, said channel being configured for receiving a nipple of said associated breast; and
a garment being coupled to each said breast engagement member, said garment comprising a flexible tubular panel, said flexible tubular panel forming a pair of recesses on an inner side of a front portion of said garment, each said recess being laterally positioned with respect to another one of said pair of recesses, said breast engagement member being positioned within one of said pair of recesses, said concave surface of said recess facing inwardly with respect to said garment, wherein said garment is configured for surrounding a back and a chest of the user, wherein said garment is configured for positioning said concave surface of each said breast engagement member in sealing engagement with one of said pair of breasts.

2. The device of claim 1, wherein said housing has an open top, said device further comprising a lid being positionable to cover said open top of said housing.

3. The device of claim 1, wherein said outer wall comprises a thermally insulating material.

4. The device of claim 2, each of said outer wall and said lid comprise a thermally insulating material.

5. The device of claim 2, wherein said lid is pivotally coupled to a back panel of said outer wall.

6. The device of claim 1, wherein said cooling unit comprises a refrigeration system.

7. The device of claim 1, wherein said container being removable from said interior space.

8. The device of claim 1, wherein said tube is coupled to said top end of said container.

9. The device of claim 1, wherein said tube extends through said lid.

10. The device of claim 1, wherein said tube extends through a front portion of said garment.

11. The device of claim 1, wherein:
said container is one of a pair of containers being positioned on said bottom panel;
said tube is one of a pair of tubes, each said tube being coupled to and extends away from an associated one of said pair of containers, each said tube having a distal end relative to said associated container, each said tube being flexible; and
said breast engagement member being one of a pair of breast engagement members, each said breast engagement member being coupled to a distal end of an associated one of said pair of tubes, each said breast engagement member having a concave surface and a channel, said channel extending through said concave surface of an associated one of said pair of breast engagement members and being fluidly coupled to said associated tube, said concave surface of each said breast engagement member being configured for sealingly engaging an associated one of a pair of breasts of a user, said channel of each said breast engagement member being configured for receiving a nipple of said associated breast, each said breast member being positioned within an associated one of said pair of recesses of said garment, said concave surface of each said breast engagement member, wherein said garment is configured for surrounding the back and the chest of the user, wherein said garment is configured for positioning said concave surface of each said breast engagement member in sealing engagement with an associated one of said pair of breasts.

12. The device of claim 11, wherein said cooling unit is laterally centered within said interior space, each said container being positioned opposite another of said pair of containers across said cooling unit, said vacuum pump being positioned above said cooling unit and between each one of said pair of containers.

13. The device of claim 1, further comprising a control circuit being said control circuit being electrically coupled to each of said cooling unit and said vacuum pump, said control circuit including a thermometer and being programmable to activate said cooling unit to cool each said container to a selected temperature, said control circuit being programmable to activate said vacuum pump to cause said negative pressure in each said container over a selected time period, at a selected frequency, and to a selected magnitude with respect to a pressure of said channel of each said breast engagement member.

14. The device of claim 13, further comprising an input being coupled to said lid for activating and programming said control circuit.

15. The device of claim 14, wherein said input comprises a plurality of buttons.

16. The device of claim 13, further comprising a display being coupled to said lid for displaying an indicia associated with operation of said automatic breast pump device.

17. An automatic breast pump device comprising:
a housing having an outer wall and an interior space, said interior space bounded by said outer wall, said housing having an open top, said outer wall comprising a thermally insulating material;
a lid being positionable to cover said open top of said housing, said lid comprising said thermally insulating material, said lid being pivotally coupled to a back panel of said outer wall;
a cooling unit being coupled to said housing, said cooling unit being positioned within said interior space for selectively cooling said interior space, said cooling unit comprising a refrigeration system, said cooling unit being laterally centered within said interior space;
a pair of containers being positioned on a bottom panel of said outer wall and within said interior space, each said container being removable from said interior space, each said container being positioned opposite another of said pair of containers across said cooling unit;
a vacuum pump being coupled to said housing, said vacuum pump being positioned within said interior space, said vacuum pump including a motor for driving said vacuum pump, said vacuum pump being fluidly coupled to a top end of each said container to selectively cause a negative pressure within each said container, said vacuum pump being positioned above said cooling unit and between each one of said pair of containers;
a pair of tubes, each said tube being coupled to and extending away from an associated one of said pair of containers, each said tube being coupled to said top end of said associated container, each said tube having a distal end relative to said associated container, each said tube being flexible, each said tube extending through said lid;
a pair of breast engagement members, each breast engagement member being coupled to said distal end of an associated one of said pair of tubes, each said breast engagement member having a concave surface and a channel, said channel extending through said concave surface of an associated one of said pair of breast engagement members and being fluidly coupled to said associated tube, said concave surface of each said breast engagement member being configured for sealingly engaging an associated one of a pair of breasts of a user, said channel of each said breast engagement member being configured for receiving a nipple of said associated breast;
a garment being coupled to each said breast engagement member, said garment comprising a flexible tubular panel, said flexible tubular panel forming a pair of recesses on an inner side of a front portion of said garment, each said recess being laterally positioned with respect to another one of said pair of recesses, each said breast engagement member being positioned within an associated one of said pair of recesses, said concave surface of each said breast engagement member facing inwardly with respect to said garment, each said tube extending through said front portion of said garment, wherein said garment is configured for surrounding a back and a chest of the user, wherein said garment is configured for positioning said concave surface of each said breast engagement member in sealing engagement with one of said pair of breasts;
a control circuit being coupled to said lid and being positioned within said interior space, said control circuit being electrically coupled to each of said cooling unit and said vacuum pump, said control circuit including a thermometer and being programmable to activate said cooling unit to cool each said container to a selected temperature, said control circuit being programmable to activate said vacuum pump to cause said negative pressure in each said container over a selected time period, at a selected frequency, and to a selected magnitude with respect to a pressure of said channel of each said breast engagement member;
an input being coupled to said lid for activating and programming said control circuit, said input comprising a plurality of buttons; and
a display being coupled to said lid for displaying an indicia associated with operation of said automatic breast pump device.
